(19) United States
(12) Patent Application Publication
(10) Pub. No.: US 2005/0233377 A1
(43) Pub. Date: Oct. 20, 2005

(54) Drug screening method for the treatment of brain damage

(76) Inventors: Mingjie Zhang, Hong Kong SAR (CN); Nikolaus J. Sucher, La Jolla, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/826,478

(22) Filed: Apr. 16, 2004

(51) Int. Cl. G01N 33/53; C12Q 1/37

(52) U.S. Cl. 435/7.1; 435/23

(57) ABSTRACT

NMR spectroscopy is used as an efficient and effective rapid screening method for identifying compounds that interact with PSD-95. This method includes the steps of taking a first NMR spectrum of a sample of free PDZ2; mixing a test compound with said sample of free PDZ2 to form a test sample; taking a second NMR spectrum of said test sample; and comparing said first and second spectra. An observable difference of the two spectra indicates that the test sample contains ingredient(s) capable of binding to the PDZ domains of PSD-95.

### FIELD OF INVENTION

The present invention is a method of screening for neuroprotective drugs. In particular, the present invention is related to screening of compounds based on the disruption of interaction between neuronal nitric oxide synthase (nNOS) and the PDZ (PSD-95; disc large and Zonula occludens-1) domains of the postsynaptic density-95 protein (PSD-95). The present invention also relates to compounds that may be used for the treatment of ischemia stroke.

### BACKGROUND OF INVENTION

N-methyl-D-aspartate receptors (NMDARs) are a subclass of ionotropic glutamate receptors that are involved in neuronal differentiation, migration, synapse formation and the shaping of axonal outgrowth patterns during the development of the central nervous system (CNS). Excessive activation of NMDARs and subsequent production of nitric oxide by nNOS contribute to neuronal damage resulting from hypoxic and ischemic insults.

Excessive production of nitric oxide by neuronal nitric oxide synthase (nNOS) subsequent to calcium influx through NMDARs is thought to be a major factor contributing to stroke-induced neuronal damage. Prevention of stroke-induced neuronal damage by direct inhibition of NMDA receptors has been shown to be problematic as NMDAR inhibitors are invariably cytotoxic. Similary, prevention of stroke-induced neuronal damage direct inhibition of nNOS is also difficult, as the activity of nNOS not associated to stroke-induced calcium influx is essential for regular neuronal function. Additionally, specific inhibitors of nNOS (ie, not inhibiting other isoforms of NOS: iNOS and eNOS) are difficult to be designed. PSD-95 represents a very attractive alternative target in inhibiting stroke-induced nNOS activation by physically uncoupling nNOS from the NMDAR. Earlier biochemical structural studies in one of the inventor's laboratory suggested that it is theoretically possible to block the nNOS binding site on the PDZ domains of PSD-95 (Tochio et al., 2000a; Tochio et al., 2000b; Tochio et al., 1999). Furthermore, it was shown that the depletion of PSD-95 in neuronal cell cultures dissociated NMDAR activity from NO production and suppressed excitotoxicity while at the same time NMDAR activity was unaffected. (Aarts et al., 2002; Sattler et al., 1999). These studies suggested that the disruption of the interaction between nNOS and PSD-95 might represent a potential therapeutic approach aimed at abating NMDAR mediated neurotoxicity. Aarts et al. also reported that a small peptide capable of blocking the interaction of NMDARs with PSD-95 were neuroprotective both in vivo.

It is an object of the present invention to provide an improved method of screening compounds for the treatment of brain damage.

### SUMMARY OF INVENTION

The present inventors set out to search for low molecular weight compounds from TCMs that are capable of binding to the PDZ domains of PSD-95 thereby disrupting its interaction with nNOS. Such compounds might be useful for the further development of therapeutic strategies based on the disruption of PDZ-based interactions of proteins (Zhang and Wang, 2003).

In accordance with one aspect of the present invention, NMR spectroscopy is used as an efficient and effective screening method for identifying compounds that interact with PSD-95. This method includes the steps of taking a first NMR spectrum of a sample of free PDZ2; mixing a test compound with said sample of free PDZ2 to form a test sample; taking a second NMR spectrum of said test sample; and comparing said first and second spectra. An observable difference of the two spectra indicates that the test sample contains ingredient(s) capable of binding to the PDZ domains of PSD-95.

In one preferred embodiment, the PSD-95 protein is first labeled with a stable isotope that is found in its polypeptide. In the most preferred embodiment, the PSD-95 protein is first uniformly labeled with ¹⁵N (or ¹³C), and the ¹H,¹⁵N (or ¹H,¹³C) HSQC (heteronuclear single quantum coherence) spectrum of ¹⁵N (or ¹³C)-labeled PSD-95 protein is used as sensitive and efficient methods for identification of compounds that are capable of binding to PSD-95.

In another preferred embodiment, the spots in the ¹H,¹⁵N HSQC spectrum shown in FIG. 1 can be used to identify compounds that disrupt, either partially or completely, the binding of the PDZ domains of PSD-95 by nNOS.

In another aspect of the present invention, a comparison of the first and second spectra may be further analyzed to identify compound that bind to only certain parts of the PDZ domains to allow specific disruption. This sensitive method uses the analysis of individual amino acids, their locations of which have been elucidated by the inventors. As an example, the chemical shift change of serine 173 (located in the second beta-strand of PSD-95 PDZ2, and is identified as a spot with chemical shifts of (1H 8.60 ppm, ¹⁵N 117.1 ppm), labeled as “S173” in FIG. 1) & alanine 231 (located in the second alpha-helix of PSD-95 PDZ, and is identified as a spot with chemical shifts of (¹H 7.78 ppm, 1⁵N 120.3 ppm), labeled as “A231” in FIG. 1) of PSD-95 PDZ domain in response to ligand binding indicate that the ligand specifically binds to the PDZ domain and the ligand binding site on the protein is a region formed by the second beta-strand and the second alpha-helix. Importantly, this ligand binding region on PSD-95 PDZ2 overlaps with the nNOS binding region on the protein (Tochio et al., 2000b, incorporated herein in its entirety), and the screening result suggest that such ligand is likely to block nNOS from binding to PSD-95.

In another embodiment, the present method may be used to screen a large number of compounds (e.g., compound libraries) by mixing various extracts or compounds together (or using complex extracts, as illustrated in the examples below), and using the present chemical shift perturbation assay. Thereafter, a test sample that contains a positive result may be progressively simplified by testing each extract or compound separately by deconvolution until a positive extract, fraction or compound is isolated.

Using the chemical shift assay according to the present invention, compounds that bind the PSD-95 may be readily identified. Using the binding site analysis, further detailed analysis may be performed on reactive compounds. For example, derivatives of naturally occurring PSD-95-binding compounds may be synthesized to check for their interaction in the active site thereof. Derivatives that have improved binding affinity may thus be readily identified.

In accordance with another aspect of the present invention, certain compounds were found from aqueous extracts of Huangqin, the root of Scutellaria baicalensis Georgi (Labiatae) bound to the second PDZ domain of PSD-95. NMR chemical shift perturbation assay demonstrated that the herb contains compounds that specifically bind to the nNOS/NR2B-binding pocket of PDZ2. Four flavones baicalin, norwogonoside, oroxylin A-glucuronide (also known as oroxyloside), and wogonoside were isolated and found to account for the PDZ-binding activity of the extract. Baicalin and norwogonoside displayed the highest PDZ2 binding affinity. The molecules identified in this study are useful as therapeutic compounds and may also provide leads for the structure-based development of drugs capable of interfering with PDZ domain-mediated signaling pathways.

The exquisite sensitivity of binding-induced chemical shift changes of PDZ2 of PSD-95 as described above provided a means for analyzing PDZ-binding components in aqueous herbal extracts by simply comparing the ¹H-¹⁵N spectra of extract-added PDZ2 with that of free PDZ2. The compounds identified in this invention serve as potential drug leads targeting the signaling pathways mediated by PSD-95 PDZ domains and represent an alternative therapeutic approach for reducing damage caused by NMDAR related neurotoxicity.

### BRIEF DESCRIPTION OF FIGURES

FIG. 1 shows a ¹H,¹⁵N HSQC spectrum of free PSD-95 PDZ2. Each backbone resonance is labeled with corresponding amino acid residue name (in one letter code) and residue number.

FIG. 2 shows the ¹H,¹⁵N HSQC spectrum of PDZ2 after incubation with a peptide (cypin peptide) derived from the PSD-95 binding protein called cypin.

FIG. 3 shows an overlay plot of FIGS. 1 and 2. In this overlay plot, the HSQC spectrum of free PSD-95 PDZ2 is drawn in black, and the spectrum of cypin peptide-bound protein is drawn in gray.

FIG. 4 shows ¹H ¹⁵N HSQC spectrum of PDZ2 of PSD-95 induced by the addition of 0.4 mg dry weight of an aqueous extract of Radix Scutellariae 9.

FIG. 5 shows ¹H ¹⁵N HSQC spectrum of PDZ2 of PSD-95 induced by the addition of 2 mg dry weight of an aqueous extract of Radix Scutellariae.

FIG. 6 shows ¹H ¹⁵N HSQC spectrum of PDZ2 of PSD-95 induced by the addition of 4 mg dry weight of an aqueous extract of Radix Scutellariae.

FIG. 7 shows an overlay plot of ¹H ¹⁵N HSQC spectra of free PDZ2 (black circles) incubated with increasing amounts of Radix Scutellariae extract. The spectra of the protein mixed with increasing amount of Radix Scutellariae extract are drawn in decreasing scale of gray color. For clarity, the movements of selected amino acid residues as a function of increasing of Radix Scutellariae extract are indicated with pointed arrows.

FIG. 8 shows the dose-response curve of the chemical shift changes of backbone amide of serine 173 and alanine 231 of PDZ2 of PSD-95 induced by the aqueous extract of Radix Scutellariae.

FIG. 9 shows an HPLC chromatogram of aqueous extracts of Radix Scutellariae.

FIG. 10 shows the chemical structure of baicalin.

FIG. 11 shows the chemical structure of oroxylin A-glucuronide (oroxyloside).

FIG. 12 shows the chemical structure of wogonoside.

FIG. 13 shows the chemical structure of nor-wogonoside.

FIG. 14 shows ¹H ¹⁵N HSQC spectrum of PDZ2 of PSD-95 incubated with purified baicalin.

### DETAILED DESCRIPTION

The present invention relies on exquisite sensitivity of NMR resonances (in the term of chemical shifts) of target proteins in response to binding of potential ligands. It provides a direct binding assay between any small molecular weight compounds with the target protein PSD-95. The method does not require any previous knowledge of the biological activity of compounds to be screened, nor any biological activity of the target protein. Since the screen relies on direct interaction between compounds and the target protein, the screening method is essentially error free.

In some embodiments, the screening method may be performed using polypeptides consisting of, consisting essentially of, or comprising the rat PSD-95 protein of SEQ ID NO: 2, the rat PDZ2 domain of SEQ ID NO: 4, the human PSD-95 protein of SEQ ID NO: 6, the human PDZ2 domain of SEQ ID NO: 8 or polypeptides consisting of, consisting essentially of, or comprising a polyeptide sequence with at least about 50%, at least about 60%, at least about 70%, at least about 80%, at least about 90%, at least about 95% or more than 95% amino acid identity to the rat PSD-95 protein, the rat PDZ2 domain, the human PSD-95 protein or the human PDZ-2 domain. Amino acid identity may be determined using any conventional software, including BLAST with the default parameters. A polypeptide consisting essentially of the PSD-95 protein or a sequence having a particular level of amino acid identity with the PSD-95 protein may have additional amino acids or fewer amino acids than the sequences of SEQ ID NO: 2 or SEQ ID NO: 6 and preferably has the ability to couple nNOS to NMDAR. A polypeptide consisting essentially of the PDZ-2 domain or a sequence having a particular level of amino acid identity with the PDZ-2 domain may have additional amino acids or fewer amino acids than the sequences of SEQ ID NO: 4 or SEQ ID NO: 8 and preferably has the ability to bind one or more ligands of the PDZ-2 domains of SEQ ID NO: 4 or SEQ ID NO: 8. The screening method may also be employed using polypeptides encoded by a nucleic acid sequence which hybridizes to SEQ ID NO: 1, SEQ ID NO: 3, SEQ ID NO: 5, or SEQ ID NO: 7 under stringent or moderate conditions. As used herein, “stringent conditions” means hybridization to filter-bound nucleic acid in 6×SSC at about 45° C. followed by one or more washes in 0.1×SSC/0.2% SDS at about 68° C. Other exemplary stringent conditions may refer, e.g., to washing in 6×SSC/0.05% sodium pyrophosphate at 37° C., 48° C., 55° C., and 60° C. as appropriate for the particular probe being used. As used herein, “moderate conditions” means hybridization to filter-bound DNA in 6× sodium chloride/sodium citrate (SSC) at about 45° C. followed by one, preferably 3-5 washes in 0.2×SSC/0.1% SDS at about 42-65° C.

The following examples further illustrate how the chemical shift perturbation technology according to the present invention may be used to identify potential compounds, even in complex herb mixtures, that can specifically interact with PDZ domains of PSD-95.

### EXAMPLE 1

### Recombinant Expression and Stable Isotope Labeling of PSD-95

To enhance the screening specificity and sensitivity, the PDZ domain of PSD-95 is uniformly labeled with ¹⁵N using a recombinant protein production method. PDZ proteins were expressed (Tochio et al. 2000a) with slight modifications. Briefly, the pET14b plasmids containing PDZ2 of PSD-95 were transformed into Escherichia coli BL21 (DE3) by electroporation. Single colonies were inoculated into LB medium and grown overnight at 37° C. in an environmental shaker. The saturated bacterial solution was used to inoculate a larger volume of LB medium (1:100 ratio) and grown in an environmental shaker at 37° C. When the OD600 reached ˜0.6, isopropyl-beta-D-thiogalactopyranoside (IPTG) was added to a final concentration of 0.2 mM for induction. Four to five hours' induction with moderate shaking under 30° C. was sufficient.

For uniform labeling of PDZ2 with ¹⁵N, the bacteria were grown for protein expression in M9 minimal medium using ¹⁵NH₄Cl (1 g/L) as the sole nitrogen source. Protein expression was induced in M9 minimal medium by adding IPTG to a final concentration of 0.4 mM overnight. Cell pellets from 1 L of bacterial cultures were resuspended in 40 ml ice-cold Ni²⁺-NTA affinity column binding buffer (5 mM imidazole, 0.5 M NaCl, 20 mM Tris-HCl, pH 7.9). Cells were lysed by one or two passes through a French press, followed by brief sonication. The cell lysate was centrifuged at 39,000×g for 20 minutes to remove debris, filtered through a 0.22 μm filter unit, and the supernatant was loaded onto a Ni²⁺-NTA column. The column was washed with 100 ml of binding buffer. His-tagged PDZ2 protein was eluted with 15 ml elution buffer (1 M imidazole, 0.5 M NaCl, 20 mM Tris-HCl, pH 7.9).

The eluted protein was dialyzed extensively against a buffer containing 50 mM Tris-HCl (pH 7.5), 2 mM EDTA, and 2 mM DTT. The N-terminal His-tag was cleaved by digesting the protein with thrombin (one unit of enzyme per mg of His-PDZ2 for four hours at room temperature). The digested protein was passed through a Sephacryl-100 gel-filtration column to remove the His-tag, thrombin and other contaminants. The fractions containing PDZ2 were pooled, dialyzed against NH₄HCO₃ solution with decreasing concentration (from 5 g/4 L down to 0.5 g/4 L), and finally into double distilled water. The protein samples were freeze-dried and stored at −20° C. until their use in NMR experiments.

### EXAMPLE 2

### NMR Analysis of Free PDZ2 Protein

In this specific non-limiting example, the PDZ-domain of recombinant PSD-95 was uniformly labeled with ¹⁵N using the recombinant protein production method described. The ¹⁵N-labeled PSD-95 was expressed in and purified from E. coli bacterial cells. The NMR spectra was acquired at 30° C. on Varian Inova 500 or 750 spectrometers each equipped with a 5 mm z-shielded gradient triple resonance probes. Lyophilized ¹⁵N-labeled PDZ2 protein dissolved in 100 mM potassium phosphate buffer at pH 6.0, with a pure recombinant protein concentration of ˜0.1-0.2 mM (typically 0.5 ml in 100 mM phosphate buffer) for spectral analysis.

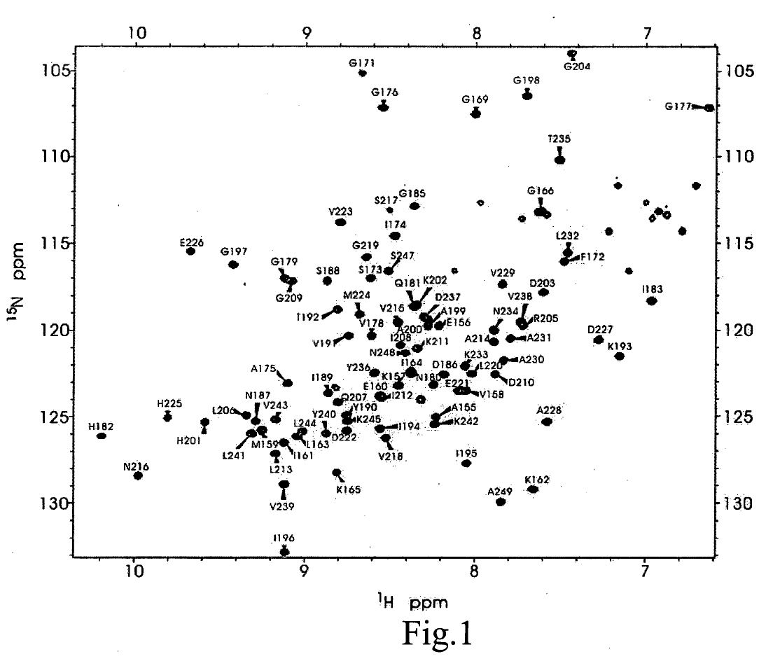

A ¹H-¹⁵N HSQC (heteronuclear single quantum coherence) with free PDS domains of PSD-95 spectrum was recorded as shown in FIG. 1. Since every amino acid residue (and only amino acid residues) in the protein is labeled with ¹⁵N, we are able to observe one resonance for each amino acid residue in the recorded ¹H-¹⁵N HSQC spectrum of the target protein. The identity of each resonance has been elucidated in our earlier experiments (Tochio et al., 2000a). The amino acid residues on PSD-95 PDZ2 that are directly involved in binding to nNOS have also been identified (Tochio et al., 2000a,b). These work laid down the foundation for the current screening of small molecular weight compounds that are capable of disrupting the formation of PSD-95/nNOS complex.

### EXAMPLE 3

### NMR Analysis of Bound PDZ2 Protein

A small peptide corresponding to the last 10 amino acid residues of a PSD-95 binding protein cypin (hereinafter referred to as the cypin peptide) was used as a test molecule to determine whether NMR may be used as a sensitive screening method according to the present invention. The cypin peptide was selected as the test molecule in these experiments because it was previously reported to actively bind the PDZ2 (Long et al., 2003; Long, J., Tochio, H., Wang, P., Fan, J.-S, Sala, C., Niethammer, M., Sheng, M. and Zhang, M. (2003) “Supramodular Structure and Synergistic Target Binding of the N-terminal Tandem PDZ Domains of PSD-95” J. Mol. Biol. 327, 203-214.).

Since the identity of every resonances of the ¹H-¹⁵N HSQC spectrum of PSD-95 PDZ domains is known, and the three dimensional structures of the PSD-95 PDZ domains could be elucidated, the quantitative values of compound-induced chemical shift changes for each amino acid residues of PSD-95 PDZ2 were computed using the equation:
Δₚₚₘ=[(Δδ_{HN})²+(Δδ_{N}·α_{N})²]^{1/2}

The scaling factor α_{N} used to normalize ¹H and ¹⁵N chemical shifts was 0.17. Δδ_{HN} corresponded to the change of the ¹⁵N-bonded hydrogen chemical shift of a specific resonance, while Δδ_{N} corresponded to the change of the ¹⁵N chemical shift of that resonance.

The value of chemical shift changes for each amino acid residue was further mapped onto the three dimensional structure of PSD-95 PDZ2 using a open or close shape scheme. The resulted compound-induced chemical shift change profile mapped onto the three dimensional structure of PDZ2 was used to judge the detailed binding property of the compound with the protein.

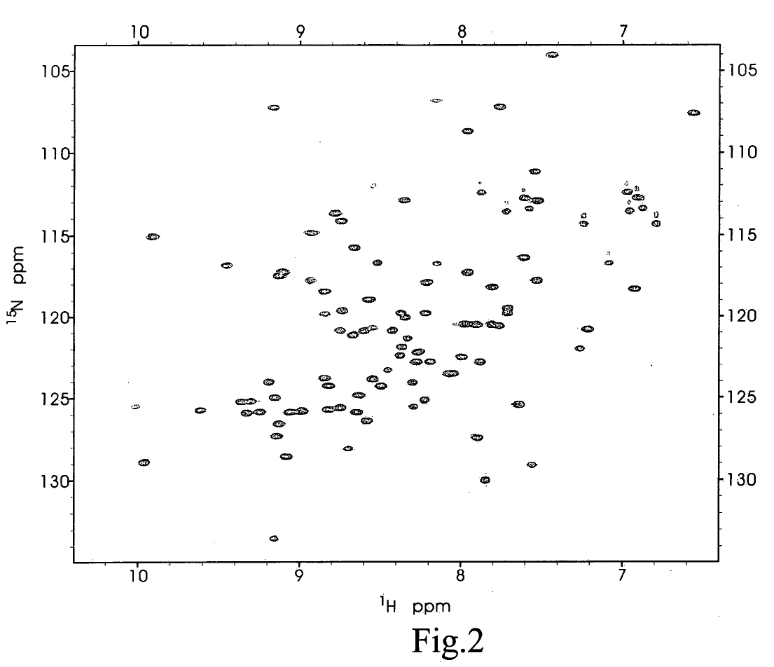

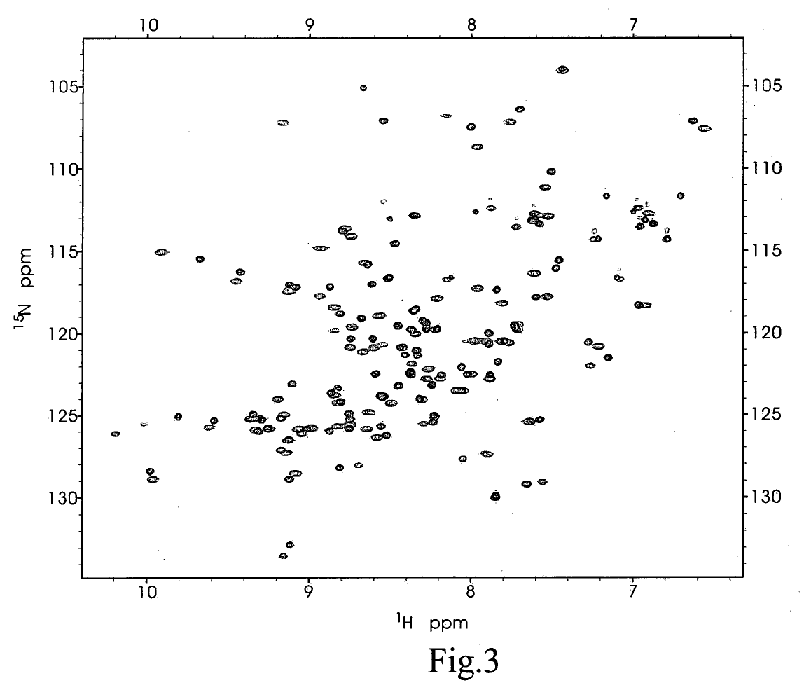

FIG. 2 shows the ¹H ¹⁵N HSQC spectrum of PSD-95 PDZ2 in the presence of the cypin peptide. As shown in FIG. 3, an overlay of FIGS. 1 & 2 shows even more clearly the chemical shift changes of PSD-95 resulted from the cypin peptide binding. For a crude analysis to determine whether there has been any binding at the PDZ domains, the computer may be set to check spots corresponding to residues serine 173 (117.1 ppm, 8.60 ppm), alanine 175 (123.2 ppm, 9.11 ppm), histidine 225 (125.1 ppm, 9.81 ppm), glutamate 226 (115.1 ppm, 9.64 ppm), alanine 228 (125.3 ppm, 7.57 ppm), and alanine 231 (120.3 ppm, 7.78 ppm) only in FIG. 1. FIG. 3 clearly shows that the binding of the cypin peptide causes a chemical shift change of amino acid residues located in the second beta-strand and in the second alpha-helix of PSD-95 PDZ2 (including the amino acid residues mentioned above) that are directly involved in the ligand binding.

The high-resolution solution structure of the second PDZ domain of PSD-95 was determined and the backbone dynamics of the PDZ domain was studied in detail by ¹⁵N-relaxation experiments using a model-free approach. The PSD-95 PDZ2 contains a long-rigid loop connecting the βB and βC strands, and this unique loop is directly involved in target binding (Tochio et al., 2000a). We computed the quantitative values of chemical shifts changes of each amino acid residue as a result of ligand binding, and mapped such ligand binding-induced chemical shift changes to the three dimensional structure of the protein (see (Tochio et al., 2000a) for example). The binding induced chemical shift changes were restricted to βB strand and αB groove of this PDZ domain of PSD-95.

### EXAMPLE 4

### Screening of Herbal Mixture for Active Compounds

Since the data in Example 3 showed that the chemical shift perturbation method of the present invention is useful and effective as a screening method, the present inventors proceeded to determine whether the present method may be used to identify potential compounds in very complex herbal mixtures. Twenty traditional Chinese medicines, as shown in Table 1, frequently used in stroke therapy (Gong and Sucher, 1999; Sun et al., 2003) were screened for compounds that might be capable of binding to PDZ2 of PSD-95.

TABLE 1
Traditional Chinese medicines used in stroke therapy and in screening for
NMDAR antagonist properties

| No. | Medicinal name | Scientific name (Family name) | Pinyin/Chinese |
|---|---|---|---|
| 1 | Rhizoma Ligustici | Ligusticum chuanxiong Hort. (Umbelliferae) | Chuanxiong [symbol] |
| | chuangxiong* | | |
| 2 | Radix Rehmanniae | Rehmannia glutinosa Libosch. (Scrophylariaceae) | Dihuang [symbol] |
| | Rhizoma Seu Radix | Notopterygium incisium Ting ex H. T. Chang | |
| 3 | Notopterygii | (Umbelliferae) | Qianghuo [symbol] |
| 4 | Cornu Saigae Tataricae | Saiga tatarica Linaeus (Bovidae) | Lingyangjiao [symbol] |
| 5 | Radix Paeoniae Rubra | Paeonia lactiflora Pall. (Ranunculaceae) | Chishao [symbol] |
| 6 | Pheretima | Pheretima aspergillum E. Perrier (Megascolecidae) | Dilong [symbol] |
| | | Scolopendra subspinipes mutilans L. Koch | |
| 7 | Scolopendra | (Scolopendridae) | Wugong [symbol] |
| 8 | Poria | Poria cocos (Schw.) Wolf (Polyporaceae) | Fuling [symbol] |
| | | Saposhnikovia divaricata (Turcz.) Schischk. | |
| 9 | Radix Saposhnikoviae | (Umbelliferae) | Fangfeng [symbol] |
| 10 | Bombyx Batryticatus | Bombyx mori L. (Bombycidae) | Jiangcan [symbol] |
| 11 | Radix Gentianae Macrophyllae | Gentiana macrophylla Pall. (Gentianaceae) | Qinjiao [symbol] |
| | | Angelica pubescens Maxim. f. biserrata Shan et Yuan | |
| 12 | Radix Angelicae Pubescentis | (Umbelliferae) | Duhuo [symbol] |
| | | Asarum heterotropoides Fr. Var. mandshuricum | |
| 13 | Herba Asari | (Maxim.) Kitag. (Aristolochiaceae) | Xixin [symbol] |
| 14 | Periostracum Cicadae | Crytotympana pustulata Fabricius (Cicadidae) | Chantui [symbol] |
| 15 | Rhizoma Gastrodiae | Gastrodia elata Bl. (Orchidaceae) | Tianma [symbol] |
| 16 | Radix Achyranthis Bidentatae | Achyrantes bidentata Bl. (Amaranthaceae) | Niuxi [symbol] |
| 17 | Flos Carthami | Carthamus tinctorius L. (Compostiae) | Honghua [symbol] |
| 18 | Radix Scutellariae | Scutellaria baicalensis Georgi. (Labiatae) | Huangqin [symbol] |
| 19 | Ramulus Uncariae cum Uncis | Uncaria rhynchophylla (Miq.) Jacks. (Rubiaceae) | Gouteng [symbol] |
| 20 | Radix Stephaniae Tetrandrae | Stephania tetrandria S. Moore (Menispermaceae) | Fangji [symbol] |

All TCMs shown above are listed in the Pharmacopoeia of the People's Republic of China [18] except the one marked with an asterisk (*).

In this method, the PDZ domain of PSD-95 is expressed recombinantly as described in Example 1 and uniformly labeled with ¹⁵N using a recombinant protein production method. The ¹⁵N-labeled PSD-95 was expressed in and purified from E. coli bacterial cells. Pure recombinant protein at concentration 0.1-0.2 mM (0.5 mL in 100 mM phosphate buffer, pH 6.0) mixed with TCM herbal extract or purified compounds from these herbs.

Using preparation techniques mentioned above, a ¹H,¹⁵N HSQC spectrum was recorded for each mixture of bound PDZ2 protein, and compared with the ¹H,¹⁵N HSQC spectrum of free PSD-95 PDZ2 (˜0.1 mM in the same buffer). NMR based chemical shift perturbation assays were used to monitor the binding of compounds from herb extracts to ¹⁵N-labeled PDZ2 of PSD-95.

The observation of chemical shift changes (with respect to the free PDZ2) as a result of mixing the protein with the herbal mixtures or pure compounds was scored as positive binding of compound(s) to PDS-95 PDZ2.

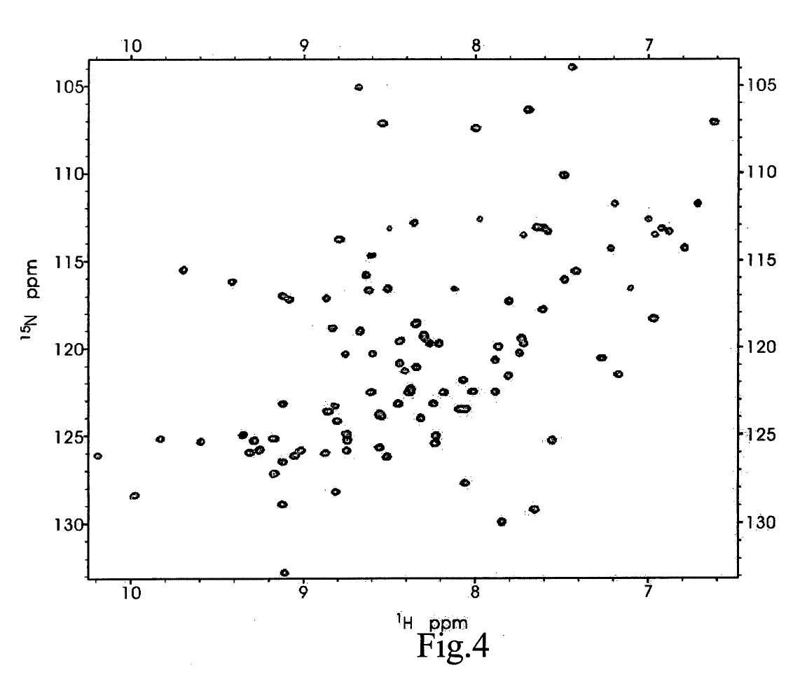

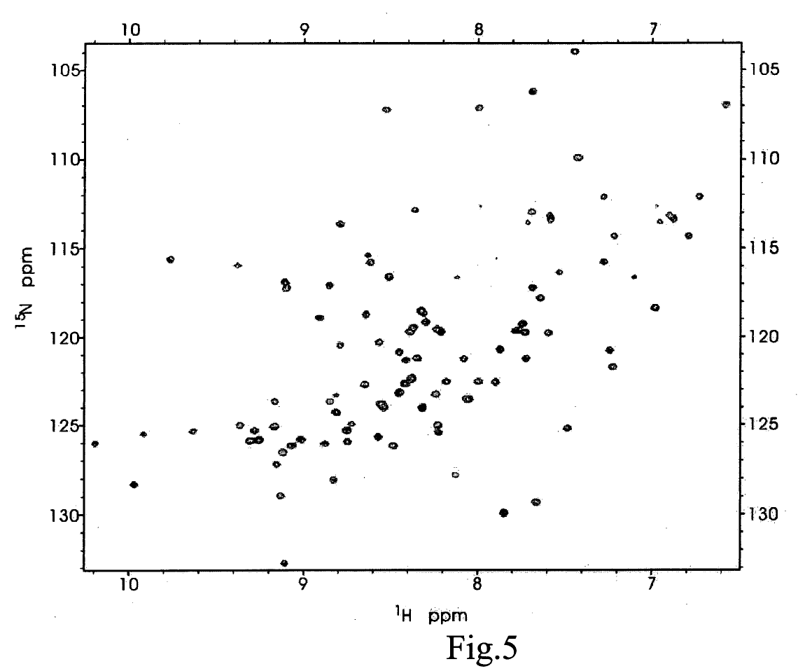

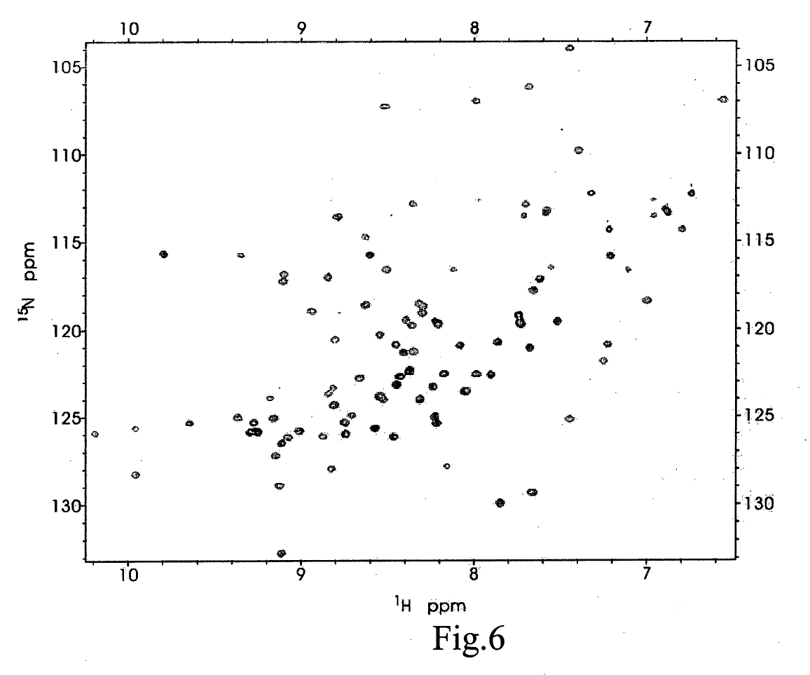

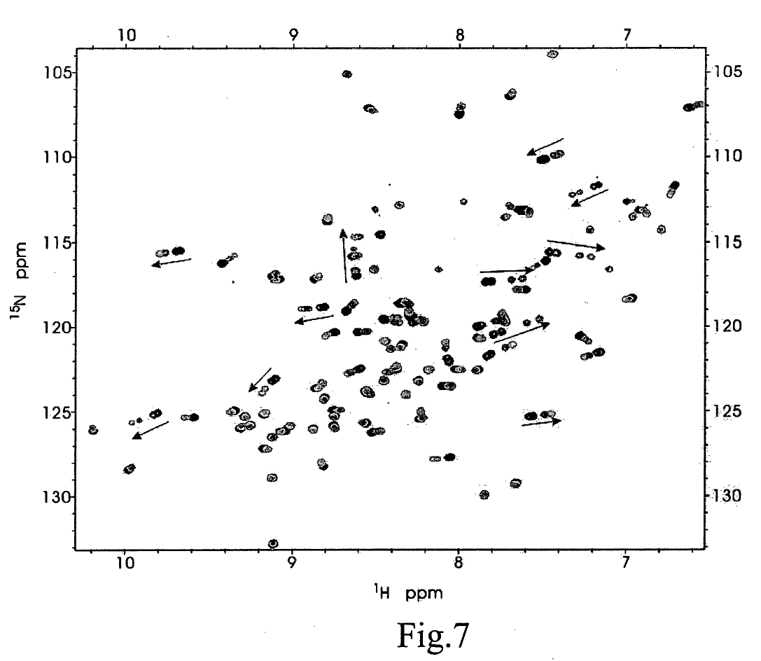

Using this approach, we found that all the Chinese medicinal showed negative results except the aqueous extract of Radix Scutellariae (compound No. 18 in Table 1) induced significant chemical shift changes to PSD-95 PDZ2. The plots of the ¹H-¹⁵N HSQC (heteronuclear single quantum coherence) spectra of PDZ2 of PSD-95 with 0.4 mg, 2 mg and 4 mg aqueous extract of Radix Scutellariae are shown in FIGS. 4, 5 and 6 respectively. FIG. 7 shows an overlay plot of the ¹H-¹⁵N HSQC (heteronuclear single quantum coherence) spectra of PDZ2 of PSD-95 with increasing concentrations of the aqueous extract of Radix Scutellariae. The compounds in the extract of Radix Scutellariae exhibited a dose dependent behaviour binding to the PDZ2 of PSD-95.

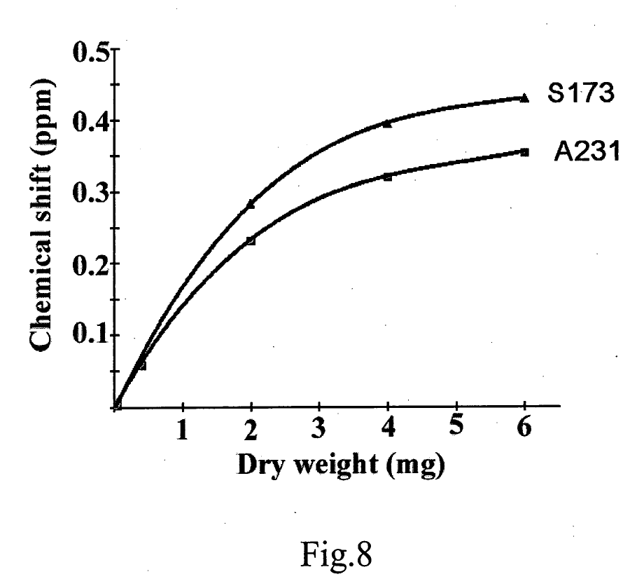

To further characterize the interaction between the compound(s) from Radix Scutellariae and PDZ2 of PSD-95, a dose-response curve, as shown in FIG. 8, was constructed by plotting the magnitude of the chemical shift changes of the amides of serine 173 (S173) and alanine 231 (A231). S173 and A231 reside in βB strand and αB helix of the protein, respectively, in relation to the dry weight of the extract. The titration curves display both dose dependent and saturable characteristics, indicative of specific interactions between the compound(s) and PDZ2 of PSD-95. Taken together, the NMR-based screening data suggested that one or more compounds in the aqueous extract of Radix Scutellariae bound specifically to the PDZ2 binding groove, resulting in the perturbation of its structure.

### EXAMPLE 5

### Identifying Active Compounds in Herbal Mixtures

Radix Scutellariae was purchased in the markets of Weichang, Hebei province, China. Ni²⁺-NTA beads were purchased from Novagen Inc. (Madison, Wis., USA). ¹⁵N labeled ammonium chloride was purchased from Cambridge Isotope Laboratories, Inc. (Andover, Mass., USA). Thrombin and DMSO were from Sigma Chemical Co. (St. Louis, Mo., USA). Sephadex™ LH-20 was purchased from Amersham Pharmacia Bioscience Inc. (Piscataway, N.J., USA). The membrane used for dialysis was Spectra/Por®, MWCO: 6-8,000, from Spectrum Laboratories, Inc. (Rancho Dominguez, Calif., USA). Penicillin was purchased from Gibco BRL, Inc. (MD, USA). IPTG was from USB™ (Cleveland, Ohio, USA). The organic solvents, such as acetyl acetate, acetonitrile, methanol, and acetic acid, and Silica Gel 60 F250 for thin layer chromatography were purchased from Merck (Darmstadt, Germany). Syringe driven filter units were Millex®-GS of Millipore Corporation (Bedford, Mass., USA).

Ten grams of Radix Scutellariae were cut into small pieces, and extracted with 100 ml ddH₂O by boiling in a Soxhlet apparatus for four hours. Fresh extract was used for testing of PDZ2 perturbation ability. For use in the experiments, the extract was either passed through 0.45 μm filter units or centrifuged at 14,000 rpm for 5 minutes to remove any precipitate.

Purification of Chemical Constituents from Radix Scutellariae

One hundred grams of Radix Scutellariae were boiled in 400-500 ml of water twice (one hour each). The extract was then filtered with gauze. The pH of the extract (˜pH 4.8) was adjusted to pH 2 with 1 M HCl, upon which a large amount of dark yellow precipitate formed. The aqueous extract was heated to 85° C. for half an hour to facilitate the precipitation of baicalin, and filtered. The precipitate was redissolved in methanol. The resulting solution was evaporated slowly to aid the crystallization of baicalin. After boiling and filtering, the aqueous extract was concentrated to 300 ml in a rotary evaporator (Bibby RE2000, Bibby Sterilin LTD, UK), and fractionated with ethyl acetate (4-5 times with 200 ml each). The ethyl acetate layer was collected and concentrated to a final volume of 20 ml. A large amount of a yellow precipitate formed. The precipitate was filtered, dried and approximately 0.5 g of orange-red powder were obtained. The powder was redissolved in 20 ml methanol/water (2:1), upon which a large amount of yellow precipitate formed. After the mixture was filtered, a ˜5 ml were loaded onto a Sephadex™ LH-20 column (inner diameter: 1.5 cm, length: 1 m), which had been equilibrated with 50% methanol in water. The column was eluted with the equilibration solution at a flow rate of ˜0.5 ml/min. Analytical thin layer chromatography (TLC) was conducted on silica gel 60 F250 precoated aluminum foil to determine the major contents of the eluate. TLC plates were developed with chloroform:methanol:H₂O (65:35:10). Bands were visualized under UV (254 nm and 365 nm) or sprayed with 1% FeCl₃ solution and heated to 110° C.

Preparative high-performance liquid chromatography (HPLC; Waters PrepLC 4000 System, Waters, USA) was used for the milligram-scale preparation of the components in Radix Scutellariae. The solvents were filtered through 0.2 μm Millipore filter membrane and degassed with helium prior to use. The conditions used in HPLC are listed in Table 2. Following filtration with 0.22 μm syringe filter units, 2 ml of the crude extract was loaded on the column. The column was eluted according to condition HPLC (1) listed in table 2. The eluate was collected at 1-minute intervals. The fractions were fast-frozen in liquid nitrogen, followed by lyophilization in order to remove all solvents.

TABLE 2

| | HPLC (1) | HPLC (2) | HPLC (3) |
|---|---|---|---|
| HPLC | Waters PrepLC ™ | Waters PrepLC ™ 4000 | Waters ™ 600 system |
| Apparatus | 4000 | | |
| Column | Alltima ™ C18 | Alltima ™ C18 column | Ultrasphere ™ ODS |
| | column | | column |
| Solvent | 0.1% Acetic Acid in | MeOH:H₂O:HAc = 4:1:95 | MeOH:H₂O:HAc = 4:1:95 |
| (A) | H₂O | | |
| Solvent | Acetonitrile | MeOH:H₂O:HAc = 4:190:5:5 | MeOH:H₂O:HAc = 4:190:5:5 |
| (B) | | | |
| Gradient | 0 min, 90:10; | 0 min, 50:50; | 0 min, 70:30; |
| | 50 min, 50:50; | 30 min, 70:30; | 40 min, 50:50; |
| | 100 min, 10:90 | 60 min, 10:90 | 80 min, 30:70 |
| Flow rate | | 2 ml/min | 1 ml/min |
| Detection | 280 nm | 270 nm/320 nm | 210 nm-390 nm |

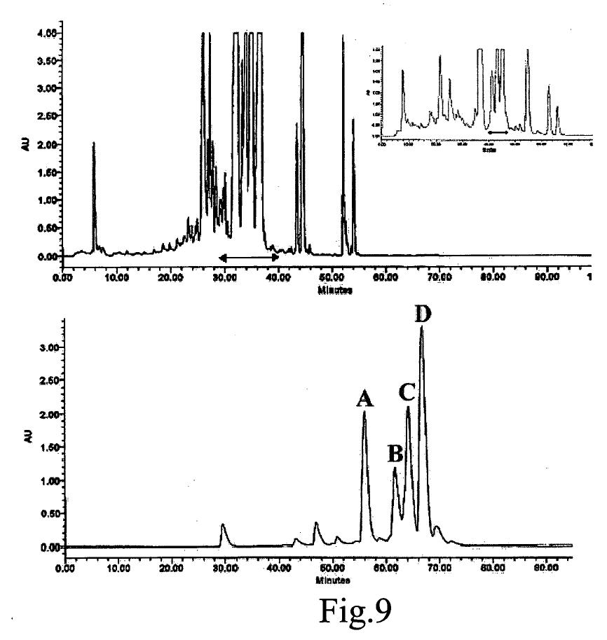

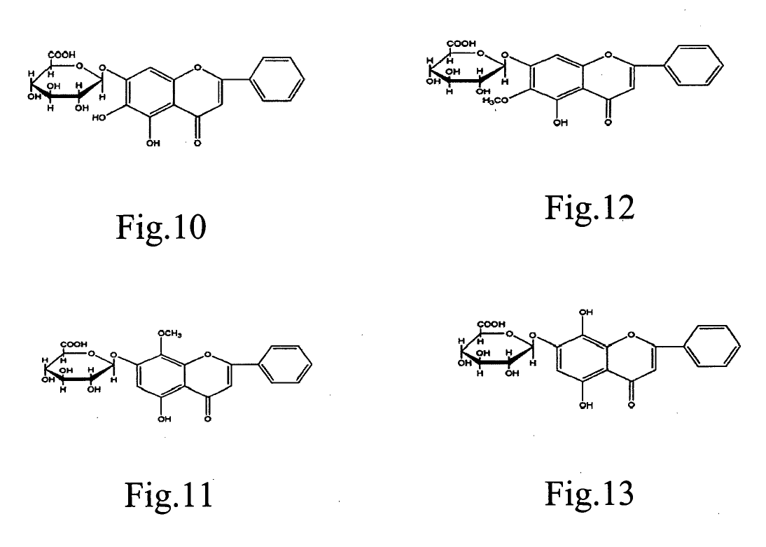

The aqueous crude extract of Radix Scutellariae was fractioned using preparative HPLC. Eventually, we obtained an active fraction from the aqueous extract of Radix Scutellariae displaying mainly four peaks in the chromatogram as shown in FIG. 9, bottom panel. The compounds accounting for the four peaks corresponded to baicalin, norwogonoside, oroxylin A-glucuronide, and wogonoside. All are flavonone 7-glucuronides differing in the substituents on ring A (FIG. 10-13). As used herein, “substantially pure” means at least 50% w/w, preferably 75% w/w and most preferably 90% w/w.

### EXAMPLE 6

### Testing Binding Affinity of Active Components

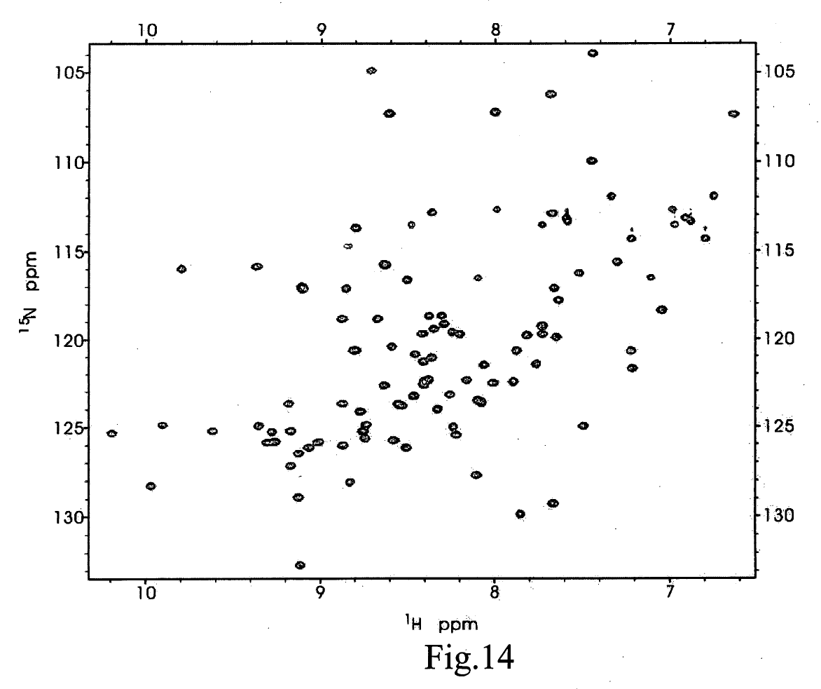

Following isolation and chemical characterization, we tested each compound for its ability to bind to PSD-95 PDZ2. All the above-mentioned fractions were tested for their ability to interact with PDZ2 of PSD-95 using the same chemical shift perturbation approach as described in Example 2. FIG. 14 shows the ¹H, ¹⁵N HSQC spectrum of PSD-95 PDZ2 incubated with purified baicalin. When compared to the ¹H, ¹⁵N HSQC spectrum of free PSD-95 PDZ2, binding of baicalin induces significant chemical shift changes to the HSQC spectrum of the protein.

In pure form, baicalin and wogonoside showed very poor solubility in H₂O. Both compounds were therefore dissolved in DMSO for the NMR titration experiments.

The binding affinity of the compounds to PDZ2 of PSD-95 was quantified by normalizing the chemical shift of the amide groups of Ser 173 and Ala 231 in relation to the maximal shift of these groups upon incubation with the crude extract (100%). Baicalin and norwogonoside bound to PDZ2 of PSD-95 with similar affinity. At a 10-fold excess of either of these compounds relative to PDZ2, the structural perturbation of PDZ2 was around 60%. However, oroxylin A-glucuronide and wogonoside were not as potent as baicalin and norwogonoside in perturbing the PSD-95 PDZ2 structure. Although the extent of the structural change of PDZ2 positively correlated with the added amount, the structural perturbation of PDZ2 only reached 10%-20% at a 10 fold excess of either of these compounds relative to PDZ2.

The flavonoid compounds identified in the examples shown above are isolated from Chinese medicinal herb extracts, but have been believed to act on the NMDA receptor directly. Until the present invention, they have not been known to interact with PSD-95. However, since these compounds have long been used for the therapeutic treatment of stroke, they are believed to be devoid of toxicity at the dosage required for the treatment, and the side effect is believed to be minimal.

It is established in this invention that the binding-induced chemical shift changes were restricted to the βB/αB-groove of this PDZ domain of PSD-95. It is important to note that the compound(s) from Radix Scutellariae are bound to the same site on PDZ2 of PSD-95 as nNOS (Tochio et al., 2000c).

The chemical shift changes of PDZ2 induced by the components in the aqueous extract of Radix Scutellariae, if compared with the peptide-induced PDZ2 chemical shift changes (Tochio et al., 2000a), are spatially more restricted. The most significant changes of PDZ2 induced by the components in Radix Scutellariae occurred in the middle of the αB/βB-groove. This perturbation profile suggests that the interaction between the compounds and PDZ2 of PSD-95 largely occurred in the center of the binding groove, and the carboxyl-binding loop and the hydrophobic pocket accommodating the side chain of the extreme C-terminal residue of peptide ligands are unoccupied. Therefore, flavonoid derivatives that might bind to PDZ2 of PSD-95 with significant higher binding affinities may be synthesized and analyzed in accordance with the present invention.

These flavanoids are not known to specifically interact with PDZ domains of PSD-95. With the experiment results provided herein, there is suggestion that these compounds may be used for the treatment of ischemia stroke.

While the present invention has been described using the examples above, these examples are non-limiting, and it is clear that one of ordinary skill in the art may provide numerous variations based on the teaching described herein. It is therefore intended that the present invention be defined by the appended claims. For example, although stable isotopes of nitrogen and carbon are used to label the recombinant protein in the aforementioned examples, it is known to those of ordinary skill in the art that the same chemical shift technology may be applied to unlabeled PSD-95.

### LIST OF REFERENCES INCORPORATED IN THEIR ENTIRETY HEREIN

1. Aarts, M., Liu, Y., Liu, L., Besshoh, S., Arundine, M., Gurd, J. W., Wang, Y. T., Salter, M. W., and Tymianski, M. (2002). Treatment of ischemic brain damage by perturbing NMDA receptor-PSD-95 protein interactions. Science 298, 846-850
2. Zhang, M., and Wang, W. (2003). Organization of signaling complexes by PDZ-domain scaffold proteins. Acc Chem Res 36, 530-538.
3. Gong, X., and Sucher, N.J. (1999). Stroke therapy in traditional Chinese medicine (TCM): prospects for drug discovery and development. Trends Pharmacol Sci 20, 191-196
4. Sattler, R., Xiong, Z., Lu, W.-Y., Hafner, M., MacDonald, J. F., and Tymianski, M. (1999). Specific coupling of NMDA receptor activation to Nitric Oxide neurotoxicity by PSD-95 protein. Science 284, 1845-1848
5. Sun, X., Chan, L. N., Gong, X., and Sucher, N.J. (2003). N-methyl-D-aspartate receptor antagonist activity in traditional Chinese stroke medicines. Neurosignals 12, 31-38.
6. Tochio, H., Hung, F., Li, M., Bredt, D. S., and Zhang, M. (2000a). Solution structure and backbone dynamics of the second PDZ domain of postsynaptic density-95. J Mol Biol 295, 225-237.
7. Tochio, H., Mok, Y. K., Zhang, Q., Kan, H. M., Bredt, D. S., and Zhang, M. (2000b). Formation of nNOS/PSD-95 PDZ dimer requires a preformed beta-finger structure from the nNOS PDZ domain. J Mol Biol 303, 359-370.
8. Tochio, H., Zhang, Q., Mandal, P., Li, Ming and Zhang, M. (1999). Solution structure of the extended neuronal nitric oxide synthase PDZ domain complexed with an associated peptide. Nat Struct Biol 6, 417-421.

1. A method of screening for compounds comprising the steps of
a) obtaining a sample of free Postsynaptic density-95 protein;
b) taking a first NMR spectrum of the free PDZ domain of said free Postsynaptic density-95 protein;
c) adding a test compound into said sample of free Postsynaptic density-95 protein to form a test sample;
d) incubating said test compound with said PDZ domain to allow binding reaction;
e) taking a second NMR spectrum of said incubated PDZ domain;
f) comparing said first and second NMR spectra wherein binding reaction is identified.

2. A method according to claim 1 wherein said method further comprises the step of:
(g) identifying test samples that cause chemical shift changes to amino acid residues in the second α-helix and the second P-strand of PSD-95 PDZ2.

3. A method according to claim 2 wherein said amino acid in step (e) is at least one amino acid selected from the group consisting of glycine 169-alanine 175 in the second β-strand and histidine 225 to lysine 233 the second α-helix of PSD-95 PDZ2.

4. A method according to claim 1 wherein said test sample contains flavonoids.

5. A method according to claim 2 wherein said test sample contains flavonoids.

6. A method according to claim 3 wherein said test sample contains flavonoids.

7. A method according to claim 1 wherein said test sample contains a crude herbal extract.

8. A method according to claim 7 further comprising separating the components of said crude herbal extract; and repeating steps (a) to (d) to identify active ingredients therein.

9. A method according to claim 5 wherein said crude extract is an aqueous extract.

10. A method of treatment of brain damage in humans resulting from hypoxic or ischemic insults comprising administering an effective dose of baicalin, oroxylin A-glucuronide (oroxyloside), wogonoside or nor-wogonoside.

11. A pharmaceutical composition comprising a substantially pure form of at least one flavanoid selected from a group comprising baicalin, oroxylin A-glucuronide (oroxyloside), wogonoside and nor-wogonoside.

12. The method of claim 1, wherein said Post-synaptic density 95 protein comprises the sequence of SEQ ID NO: 2.

13. The method of claim 1, wherein said Post-synaptic density 95 protein comprises a polypeptide with at least 50% amino acid identity to the sequence of SEQ ID NO:2.

14. The method of claim 1, wherein said Post-synaptic density 95 protein comprises the sequence of SEQ ID NO: 6.

15. The method of claim 1, wherein said Post-synaptic density 95 protein comprises a polypeptide with at least 50% amino acid identity to the sequence of SEQ ID NO: 6.

16. The method of claim 1, wherein said Post-synaptic density 95 protein is encoded by a nucleic acid which hybridizes under stringent conditions to a nucleic acid comprising a sequence selected from the group consisting of SEQ ID NO: 1 and SEQ ID NO: 5.

17. A method of screening for compounds comprising the steps of
obtaining a sample comprising the PDZ-2 domain of Post-synaptic density 95 protein;
taking a first NMR spectrum of said PDZ-2 domain of Post-synaptic density 95 protein;
adding a test compound into said sample comprising the PDZ domain of Post-synaptic density 95 protein to form a test sample;
incubating said test compound with said PDZ domain to allow a binding reaction;
taking a second NMR spectrum of said incubated PDZ domain; and
comparing said first and second NMR spectra wherein said binding reaction is identified.

18. The method of claim 17, wherein said PDZ domain comprises the sequence of SEQ ID NO: 4.

19. The method of claim 17, wherein said PDZ domain comprises a polypeptide with at least 50% amino acid identity to the sequence of SEQ ID NO: 4.

20. The method of claim 17, wherein said PDZ domain comprises the sequence of SEQ ID NO: 8.

21. The method of claim 17, wherein said PDZ domain comprises a polypeptide with at least 50% amino acid identity to the sequence of SEQ ID NO: 8.

22. The method of claim 17, wherein said PDZ domain is encoded by a nucleic acid which hybridizes under stringent conditions to a nucleic acid comprising a sequence selected from the group consisting of SEQ ID NO: 3 and SEQ ID NO: 7.


SEQUENCE LISTING (8 sequences)

SEQ ID NO: 1 - DNA, 2115 bp, Rattus norvegicus
  CDS (1)...(2115): PSD-95
atggactgtctctgtatagtgacaaccaagaaataccgctaccaagatgaagacacgccccctctggaacacagcccggcccacctccccaaccaggccaattctccccctgtgattgtcaacacggacaccctagaagccccaggatatgagttgcaggtgaatggaacagagggggagatggagtatgaggagatcacattggaaaggggtaactcaggtctgggcttcagcatcgcaggtggcactgacaacccgcacatcggtgacgacccgtccatttttatcaccaagatcattcctggtggggctgcagcccaggatggccgcctcagggtcaatgacagcatcctgtttgtaaatgaagtggatgttcgggaggtgacccattcagctgcggtggaggccctcaaagaggcaggttccatcgttcgcctctatgtcatgcgccggaaacccccagccgaaaaggtcatggagatcaaactcatcaaagggcctaaaggacttggcttcagcattgcggggggcgttgggaaccagcacatccctggagataacagcatctatgtaacgaagatcatcgaaggaggtgctgcccacaaggatggcaggttgcagattggagacaagatcctggcggtcaacagtgtggggctggaggacgtcatgcacgaggatgccgtggcagccctgaagaacacatatgacgttgtgtacctaaaggtggccaagcccagcaatgcctacctgagtgacagctatgctcccccagacatcacataccccacagccatgacccccacttcccctcggcgctactcccctgtggccaaggacctgctgggggaggaagacattccccgggaaccaaggcggatcgtgatccatcggggctccaccggcctgggcttcaacatcgtgggcggcgaggatggtgaaggcatcttcatctccttcatccttgctgggggtccagccgacctcagtggggagctacggaagggggaccagatcctgtcggtcaatggtgttgacctccgcaatgccagtcacgaacaggctgccattgccctgaagaatgcgggtcagacggtcacgatcatcgctcagtataaaccagaagagtatagtcgattcgaggccaagatccatgatcttcgggaacagctcatgaatagtagcctaggctcagggactgcatccttgcgaagcaaccccaagaggggcttctacattagggccctgtttgattacgacaagaccaaggactgcggtttcttgagccaggccctgagcttccgcttcggggatgtgcttcatgtcattgacgctggtgacgaagagtggtggcaagcacggcgggtccactccgacagtgagaccgacgacattggcttcattcccagcaaacggcgggtcgagcgacgagagtggtcaaggttaaaggccaaggactggggctccagctctggatcacagggtcgagaagactcggttctgagctatgagacggtgacccagatggaagtgcactatgctcgtcccatcatcatccttggacccaccaaagaccgtgccaacgatgatcttctctccgagttccccgacaagtttggatcctgtgtccctcatacgacacgtcctaagcgggaatatgagatagacggccgggattaccactttgtctcctcccgggagaaaatggagaaggacatccaggcacacaagttcattgaggctggccagtacaacagccacctctatgggaccagcgtccagtctgtgcgagaggtagcagagcaggggaagcactgcatcctcgatgtctcggccaatgccgtgcggcggctgcaggcggcccacctgcaccccatcgccatcttcatccgtccccgctccctggagaatgtgctagagatcaataagcggatcacagaggagcaagcccggaaagccttcgacagagccacgaagctggagcaggagttcacagagtgcttctcagccatcgtagagggcgacagctttgaagagatctatcacaaagtgaaacgtgtcattgaagacctctcaggcccctacatctgggtcccagcccgagagagactctga

SEQ ID NO: 2 - protein, 704 aa, Rattus norvegicus
  PSD-95
MDCLCIVTTKKYRYQDEDTPPLEHSPAHLPNQANSPPVIVNTDTLEAPGYELQVNGTEGEMEYEEITLERGNSGLGFSIAGGTDNPHIGDDPSIFITKIIPGGAAAQDGRLRVNDSILFVNEVDVREVTHSAAVEALKEAGSIVRLYVMRRKPPAEKVMEIKLIKGPKGLGFSIAGGVGNQHIPGDNSIYVTKIIEGGAAHKDGRLQIGDKILAVNSVGLEDVMHEDAVAALKNTYDVVYLKVAKPSNAYLSDSYAPPDITYPTAMTPTSPRRYSPVAKDLLGEEDIPREPRRIVIHRGSTGLGFNIVGGEDGEGIFISFILAGGPADLSGELRKGDQILSVNGVDLRNASHEQAAIALKNAGQTVTIIAQYKPEEYSRFEAKIHDLREQLMNSSLGSGTASLRSNPKRGFYIRALFDYDKTKDCGFLSQALSFRFGDVLHVIDAGDEEWWQARRVHSDSETDDIGFIPSKRRVERREWSRLKAKDWGSSSGSQGREDSVLSYETVTQMEVHYARPIIILGPTKDRANDDLLSEFPDKFGSCVPHTTRPKREYEIDGRDYHFVSSREKMEKDIQAHKFIEAGQYNSHLYGTSVQSVREVAEQGKHCILDVSANAVRRLQAAHLHPIAIFIRPRSLENVLEINKRITEEQARKAFDRATKLEQEFTECFSAIVEGDSFEEIYHKVKRVIEDLSGPYIWVPARERL

SEQ ID NO: 3 - DNA, 282 bp, Rattus norvegicus
  CDS (1)...(282): PDZ2 coding sequence
gaaaaggtcatggagatcaaactcatcaaagggcctaaaggacttggcttcagcattgcggggggcgttgggaaccagcacatccctggagataacagcatctatgtaacgaagatcatcgaaggaggtgctgcccacaaggatggcaggttgcagattggagacaagatcctggcggtcaacagtgtggggctggaggacgtcatgcacgaggatgccgtggcagccctgaagaacacatatgacgttgtgtacctaaaggtggccaagcccagcaatgcc

SEQ ID NO: 4 - protein, 94 aa, Rattus norvegicus
  PDZ2
EKVMEIKLIKGPKGLGFSIAGGVGNQHIPGDNSIYVTKIIEGGAAHKDGRLQIGDKILAVNSVGLEDVMHEDAVAALKNTYDVVYLKVAKPSNA

SEQ ID NO: 5 - DNA, 2304 bp, Homo sapiens
  CDS (1)...(2304): PSD-95
atgtcccagagaccaagagctcccaggtcagccctctggctcctggcacccccactgctgcggtgggcacccccactcctcacagtgctgcatagcgacctcttccaggccttgctggacatcctggactattatgaggcttccctctcagagagtcagaaataccgctaccaagatgaagacacgccccctctggagcacagcccggcccacctccccaaccaggccaattctcccccagtgattgtcaacacagataccctagaagccccaggatatgagttgcaggtgaacgggaccgagggggagatggaatacgaggaaatcacattggaaaggggtaactcaggtctgggcttcagcatcgcaggtggcactgacaacccacacatcggtgacgacccatccattttcatcaccaagatcattcctggtggggctgcggcccaggatggccgcctcagggtcaacgacagcatcctgtttgtaaatgaagtggacgtgcgcgaggtgacccactcagcggcggtggaagccctcaaagaggcaggctccatcgttcgcctctatgtcatgcgccggaagcccccggctgagaaggtcatggagatcaagctcatcaaggggcctaaaggtcttggcttcagcatcgcagggggcgtagggaaccagcacatcccaggagataatagcatctatgtaacaaagatcatcgaagggggtgctgcccacaaggatgggaggttgcagattggagacaagatcctggcggtcaacagtgtggggctagaggacgtcatgcatgaagatgctgtggcagccctgaagaacacgtatgatgttgtctacctaaaggtggccaagcccagcaatgcctacctgagtgacagctatgctcccccagacatcacaacctcttattcccagcacctggacaatgagatcagtcacagcagctacctgggcaccgactaccccacagccatgacccccacttcccctcggcgctactctccagtggccaaggacctgctcggggaggaagacattccccgagaaccgaggcgaattgtgatccaccggggctccacgggcctgggcttcaacatcgtgggtggcgaggacggtgaaggcatcttcatctcctttatcctggccgggggccctgcagacctcagtggggagctgcggaagggggaccagatcctgtcggtcaacggtgtggacctccgaaatgccagccatgagcaggctgccattgccctgaagaatgcgggtcagacggtcacgatcatcgctcagtataaaccagaagagtacagccgattcgaggccaagatccacgaccttcgggaacagctcatgaacagcagcctgggctcagggactgcgtccttgcggagcaaccccaaaaggggtttctacatcagggccctgtttgattacgacaagaccaaggactgcggcttcctgagccaggccctgagcttccgctttggggatgtgctgcatgtcatcgatgctagtgatgaggagtggtggcaggcacggcgggtccactctgacagtgagaccgacgacattgggttcatccccagcaaacggcgggttgagcgacgagagtggtcaaggttaaaggccaaggactggggctccagctctggatcgcagggtcgagaagactcggttctgagctacgagacagtgacgcagatggaagtgcactatgctcgccccatcatcatccttgggcccaccaaggaccgcgccaacgatgatcttctctccgagttccccgacaagtttggatcctgtgttccccatacgacacggcccaagcgggagtatgagatagatggccgggattaccactttgtgtcgtcccgggagaaaatggagaaggacattcaggcgcacaagttcattgaggccggccagtacaacagccacctctatgggaccagcgtccagtccgtgcgagaggtggcagagcaggggaagcactgcatcctcgatgtctcggccaatgccgtgcggcggctgcaggcggcccacctgcaccccatcgccatcttcatccgcccccgctccctggagaatgtgctagagattaacaagcggatcacagaggagcaagcccgcaaagccttcgacagagccaccaagctggagcaggagttcacagagtgcttctcagccatcgtggagggtgacagctttgaggagatctaccacaaggtgaagcgtgtcatcgaggacctctcaggcccctacatctgggttccagcccgagagagactctga

SEQ ID NO: 6 - protein, 767 aa, Homo sapiens
  PSD-95
MSQRPRAPRSALWLLAPPLLRWAPPLLTVLHSDLFQALLDILDYYEASLSESQKYRYQDEDTPPLEHSPAHLPNQANSPPVIVNTDTLEAPGYELQVNGTEGEMEYEEITLERGNSGLGFSIAGGTDNPHIGDDPSIFITKIIPGGAAAQDGRLRVNDSILFVNEVDVREVTHSAAVEALKEAGSIVRLYVMRRKPPAEKVMEIKLIKGPKGLGFSIAGGVGNQHIPGDNSIYVTKIIEGGAAHKDGRLQIGDKILAVNSVGLEDVMHEDAVAALKNTYDVVYLKVAKPSNAYLSDSYAPPDITTSYSQHLDNEISHSSYLGTDYPTAMTPTSPRRYSPVAKDLLGEEDIPREPRRIVIHRGSTGLGFNIVGGEDGEGIFISFILAGGPADLSGELRKGDQILSVNGVDLRNASHEQAAIALKNAGQTVTIIAQYKPEEYSRFEAKIHDLREQLMNSSLGSGTASLRSNPKRGFYIRALFDYDKTKDCGFLSQALSFRFGDVLHVIDASDEEWWQARRVHSDSETDDIGFIPSKRRVERREWSRLKAKDWGSSSGSQGREDSVLSYETVTQMEVHYARPIIILGPTKDRANDDLLSEFPDKFGSCVPHTTRPKREYEIDGRDYHFVSSREKMEKDIQAHKFIEAGQYNSHLYGTSVQSVREVAEQGKHCILDVSANAVRRLQAAHLHPIAIFIRPRSLENVLEINKRITEEQARKAFDRATKLEQEFTECFSAIVEGDSFEEIYHKVKRVIEDLSGPYIWVPARERL

SEQ ID NO: 7 - DNA, 292 bp, Homo sapiens
  CDS (1)...(292): PDZ2 coding sequence
gagaaggtcatggagatcaagctcatcaaggggcctaaaggtcttggcttcagcatcgcagggggcgtagggaaccagcacatcccaggagataatagcatctatgtaacaaagatcatcgaagggggtgctgcccacaaggatgggaggttgcagattggagacaagatcctggcggtcaacagtgtggggctagaggacgtcatgcatgaagatgctgtggcagccctgaagaacacgtatgatgttgtctacctaaaggtggccaagcccagcaatgcctacctgagtg

SEQ ID NO: 8 - protein, 97 aa, Homo sapiens
  PDZ2
EKVMEIKLIKGPKGLGFSIAGGVGNQHIPGDNSIYVTKIIEGGAAHKDGRLQIGDKILAVNSVGLEDVMHEDAVAALKNTYDVVYLKVAKPSNAYLS